(19) United States
(12) Patent Application Publication
(10) Pub. No.: US 2009/0047745 A1
(43) Pub. Date: Feb. 19, 2009

(54) BULK NEGATIVE INDEX OF REFRACTION MATERIALS WITH RESPONSE IN THE VISIBLE

(75) Inventors: Thomas MAGNERA, Louisville, CO (US); Josef MICHL, Boulder, CO (US)

Correspondence Address: GREENLEE WINNER AND SULLIVAN P C, 4875 PEARL EAST CIRCLE, SUITE 200, BOULDER, CO, 80301, US

(73) Assignee: THE REGENTS OF THE UNIVERSITY OF COLORADO, Denver, CO (US)

(21) Appl. No.: 12/190,723

(22) Filed: Aug. 13, 2008

Related U.S. Application Data
(60) Provisional application No. 60/955,459, filed on Aug. 13, 2007.

(51) Int. Cl. G01N 21/00; C07H 21/04; C07H 1/00

(52) U.S. Cl. 436/164; 536/22.1; 536/23.1; 536/25.3

(57) ABSTRACT

Bulk negative refractive index materials (NIM) or left-handed metamaterials (LHM) as an amorphous and isotropic solid material consisting of mutually electrically insulated synthetic rings or pucks smaller than the wavelength of visible light. Each ring or puck contains a nanometer sized metallized nucleic acid ring containing one or more non-metallized segments. The bulk material can be formed as a plurality of individual pucks randomly oriented, or as multiple rings stacked together in an axial direction whose axes may be randomly oriented.

### CROSS-REFERENCE TO RELATED APPLICATIONS

This application claims priority to U.S. provisional application Ser. No. 60/955,459, filed Aug. 13, 2007, which is hereby incorporated by reference in its entirety to the extent not inconsistent with the disclosure herewith

### STATEMENT REGARDING FEDERALLY SPONSORED RESEARCH OR DEVELOPMENT

This invention was made, at least in part, with government support from the National Science Foundation SGER program CHE-0731760. The government has certain rights in the invention.

### BACKGROUND OF THE INVENTION

Negative refractive index materials (NIM), also called left-handed metamaterials (LHM), have unusual optical properties not normally found in nature and offer potential use in a wide range of applications in subwavelength imaging. (Smith et al. “Metamaterials and Negative Refractive Index” Science, 2004, 305, 788-792; Soukoulis et al. “Negative Refractive Index at Optical Wavelengths” Science 2007, 315, 47-49; Pendry, J. B.; Holden, A. J.; Robbins, D. J., Stewart et al. “Magnetism from Conductors and Enhanced Nonlinear Phenomena” IEEE Trans. Microwave Theory and Techniques 1999, 47, 2075-2084). They are heterogeneous materials with embedded active circuit devices much smaller than the wavelength of electromagnetic radiation of interest and are called left-handed because an electromagnetic wave propagates through them with its wave vector backwards relative to the cross-product of the electric and magnetic vectors.

The most important property of these materials is that they have a negative refractive index, which can permit the perfect focusing of light below the diffraction limit. (Smith et al. Science, 2004, 305, 788-792; Shelby et al. “Experimental Verification of a Negative Index of Refraction” Science 2001, 292, 77-79; Shalaev et al. “Negative Index of Refraction in Optical Metameterials” Opt. Lett. 2005, 30, 3356-3358; Pendry, J. B. “Negative Refraction Makes a Perfect Lens” Phys. Rev. Lett. 2000, 85, 3966-3969). NIM were first considered by Veselago (“The Electrodynamics of Substances with Simultaneous Values of ε and μ” Sov. Phys. Usp. 1968, 10, 509) and recently reexamined by Pendry (Phys. Rev. Lett. 2000, 85, 3966-3969) who further proposed ways to make materials that possess these properties. Since then there has been considerable success with NIM made and tested in the spectral range from the microwave to 100 THZ. (Linden et al. “Magnetic Response of Metamaterials at 100 Terahertz” Science 2004, 306, 1351-1353). Also the principle of sub-wavelength imaging has been demonstrated to λ/3 by an Ag superlens under conditions for which it approximates a NIM. (Fang et al. “Sub-Diffraction-Limited Optical Imaging with a Silver Superlens” Science 2005, 308, 534-537). Extending the response of NIM to the optical part of the spectrum has generated intense interest and is the focus of the present disclosure. (Dolling et al. “Cutwire pairs and plate pairs as magnetic atoms for optical materials” Optics Lett. 2005, 30, 3198-3200; Zhang et al. “Experimental Demonstration of Near-infrared Negative-index Materials” Phys. Rev. Lett. 2005, 95,137404-137408; Grigorenko et al. “Nanofabricated media with negative permeability at visible frequencies” Nature, 2005, 438, 335-338; Zhou et al. “Saturation of the Magnetic Response of Split-Ring Resonators at Optical Frequencies” Phys. Rev. Lett. 2005, 95, 223902-223903).

To be a NIM or left-handed, a material requires both a negative electric permittivity ε and a negative magnetic permeability μ. Whereas useful materials with ε<0 in the visible region are commonly found in nature (metals and other strongly absorbing compounds near a resonance), materials with useful μ<0 in the visible are not known. Materials with μ<0 at high frequencies must be designed rather than found and this is accomplished by adding a component within the material that has a magnetic resonance at the desired working frequency. To date, most functioning NIM have been stacked arrays of resonant metal L-C-R (inductance-capacitance-resistance) circuits much smaller than the resonant microwave wavelength. They resemble a pile of printed circuit boards more than a true material. Currently under development are new approaches that use smaller micron or submicron-scale heterogeneous nanostructures, with the length scales needed for an optical response when embedded in a dielectric medium. (Shalaev et al. Opt. Lett. 2005, 30, 3356-3358; Dolling et al. Optics Lett. 2005, 30, 3198-3200; Zhang et al. Phys. Rev. Lett. 2005, 95,137404-137408; Gabitov et al. “Double-resonant Optical Materials with Embedded Metal Nanostructures” J. Opt. Soc. Am. B. 2006, 23, 535-542). However, they still require lithography for the formation of the active components on the surface of a substrate and do not lead to a bulk material.

The impact of inexpensive synthetically produced NIM compounds performing in the visible region would be world-wide and nearly instantaneous. There are numerous applications of such materials once these currently hypothetical materials become available. See for example U.S. Pat. Nos. 7,187,050 (Optical sensor and method of manufacturing the same); U.S. Pat. No. 7,186,266 (Bifocal intraoccular telescope for low vision correction); U.S. Pat. No. 7,075,689 (Optical Scanning unit and optical scanning device in the same); U.S. Pat. No. 7,053,986 (Projection optical system, exposure apparatus, and device); U.S. Pat. No. 6,958,729 (Phased array metamaterial antenna system); U.S. Pat. No. 6,856,377 (Relay Image optical system, and illuminating optical device and exposure system provided with the optical system); U.S. Pat. No. 6,791,432 (Left handed composite media); U.S. Pat. No. 6,788,273 (Radome compensation using matched negative index or refraction materials); U.S. Pat. No. 4,818,046 (Light beam scanning device); U.S. Pat. No. 4,226,500 (Scanning optical system with reflective lens).

The present invention discloses the chemical synthesis of a bulk NIM which does not rely on lithography.

### SUMMARY OF THE INVENTION

The present invention provides a chemical synthesis of a bulk negative refractive index material (NIM) as an amorphous and isotropic solid material comprising mutually electrically insulated synthetic pucks or rings smaller than the wavelength of visible light. The pucks or rings are formed from nucleic acid rings which are partially metallized to form multiple split rings. Availability of a bulk NIM offers increased design flexibility (e.g., spin coating) and lower cost for materials having specific desired optical properties.

The basic material from which the bulk NIM is prepared is a puck or ring containing a nano-sized electronic L-C-R circuit formed by a suitably shaped metal (e.g., silver) nanowire embedded in an organic/inorganic electrical insulator. The metallized ring structure acts as an inductor with one or more gaps in metallization providing the needed circuit capacitance.

More specifically, a puck or ring comprises a nanometer sized metallized nucleic acid ring containing one or more metallized segments and one or more non-metallized segments to form a single or multiple split metallized ring (FIG. 1 and FIG. 2). In one embodiment, a bulk NIM comprises a plurality of such single or multiple split metallized rings in which the axes of the rings are randomly oriented. A bulk NIM can comprise a plurality of single or multiple split metallized rings of the same size (e.g, ring diameter and circumference) or can comprise single or multiple split metallized rings of different sizes (e.g., different ring diameters and circumferences).

In one embodiment, a bulk negative refractive index material comprises a plurality of single or multiple split-metallized rings which are optionally stacked together in an axial direction. Stacking of two or more of such rings can improve the intensity of the response. More specifically an embodiment of the invention provides a NIM comprising two or more stacked metallized nucleic acid rings, each nucleic acid ring containing one or more unmetallized segments, wherein the negative refraction material has negative electric permittivity and negative magnetic permeability. To achieve both negative electric permittivity and magnetic permeability in the desired wavelength region, such as visible light, the material may be a composite material.

The nucleic acid of the rings of this invention may be single strand DNA, double strand DNA, RNA, chemically modified nucleic acids, or combinations thereof. The nucleic acid rings can be any size suitable to achieve the designed characteristics of the material while still able to maintain the shape and structural integrity of the ring without breaking. A nucleic acid ring is preferably nanometer sized. The nucleic acid rings can have a circumference ranging from 50 nm to 1,000 nm depending on the desired portion of the electromagnetic spectrum that the material is to be used with. For example, the ring circumference can range from 320 nm to 1000 nm (near IR), from 60 nm to 100 nm (UV), from 60 to 320 nm (UV-VIS), or from 110 to 220 nm (human vision).

In one embodiment, the rings or pucks of the present invention are much smaller than the wavelength range of a desired portion of the electromagnetic spectrum. Preferably the desired portion of the electromagnetic spectrum is visible light. In one embodiment, the rings or pucks of the present invention have a size about ⅓ the wavelength of visible light, more preferably about 1/10 the wavelength of visible light. This will allow the material to behave homogeneously. While it is preferable that the size of the rings or pucks is approximately 1/10 the wavelength of visible light, materials of the present invention can have a size on the same order as visible light and still be operable.

One embodiment of the invention provides a method of making a negative refractive index material comprising: linearly attaching one or more metallizable nucleic acid segments to one or more non-metallizable segments to form a linear nucleic acid element; joining the ends of the linear nucleic acid element to form a circular split-ring template; connecting two or more of the split-ring templates together in parallel; and metallizing two or more split-ring templates so that the metallizable nucleic acid segments are coated with a conductive metal while the one or more non-metallizable segments remain uncoated by the metal. Preferably, the lengths of the linear nucleic acid elements are approximately 50 nm to approximately 1,000 nm or any subranges thereof.

The metallized nucleic acid rings, which also contain one or more non-metallized segments, when stacked are stacked so that each ring is parallel to the other rings in the stack and the center axis of each ring is aligned with the axes of the other rings in the stack (see FIGS. 4 and 17). In one embodiment, the bulk material of the present invention is comprised of low-resistance cylindrical sheets. The stacked metallized nucleic acid rings are spaced so that the inter-ring axial spacing is small enough to allow the metallization of the nucleic acid rings to form conductive bridges between the rings. Preferably, the space between the nucleic acid rings is smaller than the radial metal thickness.

In an additional embodiment, the metallized nucleic acid split rings are coated with an insulating layer, preferably with a self-assembled monolayer such as alkanethiol. In this embodiment, all of the metallized surfaces of the nucleic acid rings are preferably coated by the insulating layer. More preferably, the metallized nucleic acid split rings, including the non-metallized portions thereof, are coated by the insulating layer.

### BRIEF DESCRIPTION OF THE DRAWINGS

FIG. 1 illustrates an exemplary chemical synthesis of nanometer-sized split-ring resonators with a single capacitor gap from two single strand DNA molecules that incorporate a segment that blocks subsequent metallization. The ends of the two ssDNA molecules are complementary to each other.

FIG. 2 illustrates an exemplary chemical synthesis of a split-ring resonator with two dielectric filled gaps.

FIG. 3 shows a graph of calculated relative permeabilities (μ′: real, top line and μ″: imaginary, bottom line) for Ag DNA-ring-L-C-R resonators with two gaps as a function of wavelength, diluted (1:5) and isotopically distributed in a dielectric matrix with resonances at 488 nm. (A) Single ring with T=3.1×10⁻¹⁴ s (outer radius: 40 nm, metal diameter 20 nm, gap distance: 7.2 nm). (B) Double ring with T=6.2×10⁻¹⁵ s, adjusted to account for scattering effects (outer radius: 40 nm, metal diameter 20×40 nm, gap distance: 4.3 nm).

FIG. 4 illustrates an exemplary method of forming pucks or stacks by linking ring pairs in one embodiment of the present invention.

FIG. 5 shows (Top) real part ε₁ of the permittivity of Ag; and (Bottom) plasmon resonances for an Ag ring (r=40 nm) for different metal thicknesses (radius: thickness).

FIG. 6 shows a summary of Method 6 (described below): (ss) solid support, (a) automated DNA synthesis, (b) formation of 5′-phosphorimidazolide from 1-ethyl-3-(N,N-diethylamino)propylcarbodiimide hydrochloride (EDC) in imidazole buffer followed by treatment with excess ethylene diamine or other diaminoalkane, (c) protected dicarboxylic acid molecular spacer, e.g. polyphenylethynylene with dicyclohexylcarbodiimide (DCC), (d) formation of blocking Yy duplex in 66 mM Tris-HCL, pH 7.6, 10 mM MgCl₂, at 80° C. followed by slow cooling (optional step), (e) deprotection of molecular spacer, e.g. R=2-trimethylsilylethyl with fluoride followed by treatment with DCC to a solution of oligos (A) and (B), (f) heat to 94° C. and cool, (g) Taq Polymerase.

FIG. 7 shows a summary of Method 7: steps (a)-(e) are the same as Method 6, (h) buffered T4 ligase at 12° C. (Top) Synthesis of oligomer A with a-5′-S-3′ coupled hydrophobic rigid rod spacer and oligomer B. (Bottom) Combination of A and B to form a single-gap ring template.

FIG. 8 shows a summary of Method 8: steps (a)-(e), and (h) are the same as previous methods, (i) application of restriction enzymes Pstl (providencia stuartii) and Sacl (streptomyces achromogenes) in a buffered solution and MgCl₂ at 37° C. to a natural or biosynthetic source of duplex DNA to make two different 3′-overhangs.

FIG. 9 shows a summary of Method 9: steps (a)-(g) are the same as previous methods. (Top) The synthesis of N A-5′-S-5′-B type oligomers following Method 6. (Bottom) The synthesis of a three-gap ring template by the combination of three different oligomers [N A with complementary end pairs Yᵢyᵢ] which form the link and act as primers for the polymerase step. Each oligomer A has two non-complementary segments (U).

FIG. 10 shows a summary of Method 10: steps (a)-(g) are the same as previous methods. (Top) The synthesis of N A-5′-S-3′-B type oligomers [N A and the synthesis of N linking oligomers] [N B following method 7]. (Bottom) The synthesis of a three gap ring template by the combination of three different Yᵢ-5′-S-3′-Zᵢ oligomers, [N A with cross-linking oligomers zᵢyᵢ₊₁].

FIG. 11 shows a summary of Method 11: steps (a), (d), (h) and (g) are the same as previous methods, (m) this is a modification of the reaction necessary for the introduction of oligonucleotides with functionalized base groups, B*during automated synthesis. (Top) The synthesis of oligomers, A with a m-bp sequence of bases B*, and complementary n-bp ends (Y, Z) and oligomer B with two complementary n-bp sequences (y, z). (Bottom) The synthesis of a single-gap ring template by the combination of oligomers A and B. The method of metallization is chosen to be selective for the m-bp sequence with the functionalized bases.

FIG. 12 shows a summary of Method 12: steps (a), (d) and (g) are the same as previous methods. (Top) The synthesis of two sets of oligomers: A and B with a m-bp sequence of functionalized bases B*, and different 4-bp ends that match the 3′ overhangs of Pstl and Sacl; C and D prepared either by application of restriction enzymes Pstl and Sacl (reaction i) or by autosynthesis with ordinary bases, B. (Bottom) The synthesis of a single-gap ring template by the combination of the sticky ends of oligomers A and B with complementary sticky ends of oligomers C and D. The functionalized bases are used as a doorway for the attachment of metallization blocking groups such as dendrimers.

FIG. 13 shows a summary of Method 13: steps (a), (d) and (m) are the same as previous methods, (n) an alkyl thiol is substituted for the alkyl amine used in similar experiments. (Top) The synthesis of oligomers A with a m-bp sequence of bases B* and complementary n-bp ends (Y, Z) and oligomer B with two complementary n-bp sequences (y, z). (Bottom) The synthesis of a single gap ssDNA ring template by the combination of oligomers A and B. The method of metallization works on ssDNA only. The alkanethiols attached to the derivatized bases by reaction n become the nucleation sites for metal nanoparticles. The underivatized segment is inert to metalization.

FIG. 14 shows a summary of Method 14: steps (d) and (h) are the same as previous methods, (o) a repeated series, e.g. for starburst dendrimers exhaustive Michael addition of methyl acrylate followed by exhaustive amidation with excess ethylenediamine. Adapted synthesis of a single-gap ring template by method 6. Dendrimer formation is shown before completion oligomer, AB. Dendrimer growth can be done after the formation of the ring template on suitable molecular spacer molecules to avoid interference with some polymerases (reaction g).

FIG. 15 shows a summary of Method 15: steps (d), (h) and (o) are the same as previous methods. The synthesis of a single-gap dsDNA ring template by adaption of Method 12. Dendrimer growth on the incorporated oligomer A blocks subsequent metallization.

FIG. 16 shows a summary of Method 16: steps (a) (d) and (h) are the same as previous methods. Synthesis of oligomers A and B follows Method 11 except that the sequence of oligomer A is specified as m repetitions of the telomer sequence T (k=6). The telomer, T is synthesized by standard methods (reaction a). (Bottom) The synthesis of a single-gap ssDNA ring template by Method 11. Exposure (reaction d) to the telomer puts binding sites for metal nanoparticles around the ring template.

FIG. 17 shows a summary of Method 17: steps (a)-(d) are the same as previous methods. (Top) Adapted synthesis of a double-gap dsDNA ring template by Method 10. Standard synthesis of oligomers, A, and adapted synthesis of oligomers [1]B and [2]B to include internally complementary 24-bp sequences for the formation of a hairpin between each unmetallizable segment. Within the hairpin of oligomer [1]B there is a 6-bp restriction enzyme recognition sequence, HP (e.g. Pstl) for making a 3′-overhang and within oligomer [2]B, the same sequence is inverted for making a complementary 3′-overhang. In each hairpin there is 4-bp sequence of thymine bases for making the end loop. (Bottom) The synthesis stacked double-gap dsDNA ring templates from two ring templates made separately by Method 10. One is the combination of oligomer A and [1]B and the second is the combination of oligomer A and [2]B. Intact hairpins are cut (reaction i) to make a pair of sticky ends in hairpins HP and hp that match complementary sticky ends on the mating ring. An extension of the method to a triple-stacked ring is also shown.

### DETAILED DESCRIPTION OF THE INVENTION

As used herein, the term “metallized” refers to a process where a molecule is coated or impregnated with a metal, alloy or a composition containing a metal as is known in the art. Preferably the metal is any metal having good conductive properties, including but not limited to gold, silver, or mixtures thereof.

As used herein, the term “metallizable” refers to a nucleic acid segment which is able to be coated or impregnated with a metal, alloy or a composition containing a metal. The term “non-metallizable” refers to a spacer molecule or inert nucleic acid segment which does not get coated or impregnated with a metal, alloy or a composition containing a metal using the methods disclosed herein or other similar methods as are known in the art. For example, inert nucleic acid spacers are segments of the nucleic acid that contain derivatized bases that do not allow metallization of the spacer segment during selective methods used to metallize the rest of the nucleic acid. Alternatively, a metallizable nucleic acid segment of the nucleic acid ring may be substituted with a spacer molecule, such as PNA (peptide nucleic acid) or a polyphenylethylene organic molecule, that is not metallizable under the methods used to metallize the rest of the ring. Similarly, spacer molecules may be molecules, such as dendrimers, attached to a nucleic acid segment which are not metalllized or block metallization of the spacer segment of the nucleic acid. The metallized nucleic acid rings of the present invention comprise one or more metallizable nucleic acid segments and one or more non-metallizable spacer molecules or inert nucleic acid segments. Methods of metallizing nucleic acid segments, both selectively and non-selectively, are known in the art. (Eichen et al. “Self-assembly of nanoelectronic components and circuits using biological templates” Acta. Polym. 1998, 49, 663-670; Braun et al. “DNA-templated assembly and electrode attachment of a conducting silver wire” Nature 1998, 391, 775-777; Richter, J. “Metallization of DNA” Physica E2003, 16, 157-173; Monson et al. “DNA-Templated Construction of Copper Nanowires” Nanoletters 2003, 3, 359-363; Patolsky et al. “Au-Nanoparticle Nanowires Based on DNA and Polylysine Templates” Angew. Chem. Int. Ed. 2002, 19, 2323-2327; Weizmann et al. “Telomerase-Generated Templates for the Growing of Metal Nanowires” Nano Letters 2004, 4, 787-792; Keren et al. “Patterned DNA Metallization by Sequence Specific Localization of a Reducing Agent” Nano Letters 2004, 4, 323-326; Keren et al. “Sequence-Specific Molecular Lithography on Single DNA Molecules” Science 2002, 297, 72-75).

Dendrimers, as are well known in the art, are regularly branched molecules made from repeating monomer subunits that may contain functional groups on the molecular surface. In starburst dendrimers, such as poly(amidoamine) dendrimer, each repeat subunit has its own branching point allowing for symmetrical branching. (Tomalia et al. “A New Class of Polymers: Starburst-Dendritic Molecules” Polym. J., 1985, 17,117-132).

Materials of this invention are prepared from metallized split rings comprising a molecular ring formed from nucleic acid, e.g., by covalent bonding of a plurality of nucleic acid bases. By “nucleic acid ring”, it is meant any closed circular or non-circular nucleic acid loop where the 5′ end is connected to the 3′ end or a loop resulting from complementary overlap. (Note: certain methods described herein overlap the 3′ ends to form loops.) Each molecular ring has a circumference and a cross sectional area. In one embodiment the ring is circular in shape, and has a diameter which defines the cross-sectional area and a center axis where the plane of the ring is perpendicular to the center axis. However, the ring can be amorphous or have the shape of another polygon, i.e., a square shaped loop. The ring can also be planar or non-planar.

The cross-sectional area of the ring determines the response strength which is linearly proportional to the area enclosed by the loop. Therefore, the loop material is most efficiently used and the metamaterial condition (λ>>device dimension) is satisfied best when the aspect ratio of the enclosed area is close to one.

As used herein, the term “stacked” refers to two or more metallized nucleic acid rings (with each nucleic acid ring containing one or more unmetallized segments) connected together so that each ring (ring plane) in a stack is parallel to each other ring and the center axis of each ring is aligned with the axes of the other rings in the stack. When stacked, the metallized nucleic acid rings thus form a cylindrical shape. Although the terms “parallel”, “perpendicular” and “aligned” are used to describe the orientation of the nucleic acid rings in a stack, one skilled in the art will appreciate that some deviation from these orientations and relative orientations is permissible without significantly affecting the characteristics and function of the stack.

Materials of this invention can contain metallized nucleic acid rings of the same circumference or different circumferences. Rings of the same circumference include those where the circumferences are within 10% of each other, and may include rings having different general shapes. When metallized nucleic acid rings of this invention are circular, they may have the same or different diameters, where rings of the same diameter are those where the diameters are with in 10% of each other. Metallized nuclei acid rings of different circumferences or different diameters are those that do not fall within the 10% defined range for circumferences or diameters that are the same. When a material with a broadband response is desired the range of circumference or diameters will not be limited to be within 10% but tailored to the designed performance of the material either by altering the distribution of circumferences, diameters and/or shapes or by combing different distributions.

The term “nucleic acid” includes both DNA and RNA without regard to molecular weight or source. Nucleic acids include the full range of polymers of single or double stranded nucleotides, including chemically modified nucleotides, as known in the art that are capable of forming base pairs, joinable with other nucleic acids, and cleavable by processes described herein. A nucleic acid typically refers to a polynucleotide molecule comprised of a linear strand of two or more nucleotides (deoxyribonucleotides and/or ribonulceotides) or variants, derivatives and/or analogs thereof. The exact size or length of nucleic acid employed depends upon the application and many other factors, as is known in the art. Nucleic acids may be derived from any natural source or may be synthetic. A DNA molecule is any DNA molecule of any size, from any source, including DNA from viral, prokaryotic and eukaryotic organisms, as well as synthetic DNA and variants, derivatives and analogs thereof. A RNA molecule is any RNA molecule of any size, from any source, including RNA from viral, prokaryotic and eukaryotic organisms, as well as synthetic RNA and variants, derivatives and analogs thereof. The RNA and DNA may be single stranded or double stranded.

### EXAMPLES

The following examples are intended to illustrate but not limit the invention.

### Chemical Synthesis

The NIMs of the present invention are built from metallized and non-metallized DNA segments formed into a loop whose function is to respond to a visible light field in a manner that makes the permeability negative through a designed resonant response. While some of the individual steps used in the synthesis of the LHMs or NIMs of the present invention may be based on known procedures in other fields, it should be noted that the literature does not describe combining these steps together to produce the device elements or materials of the present invention.

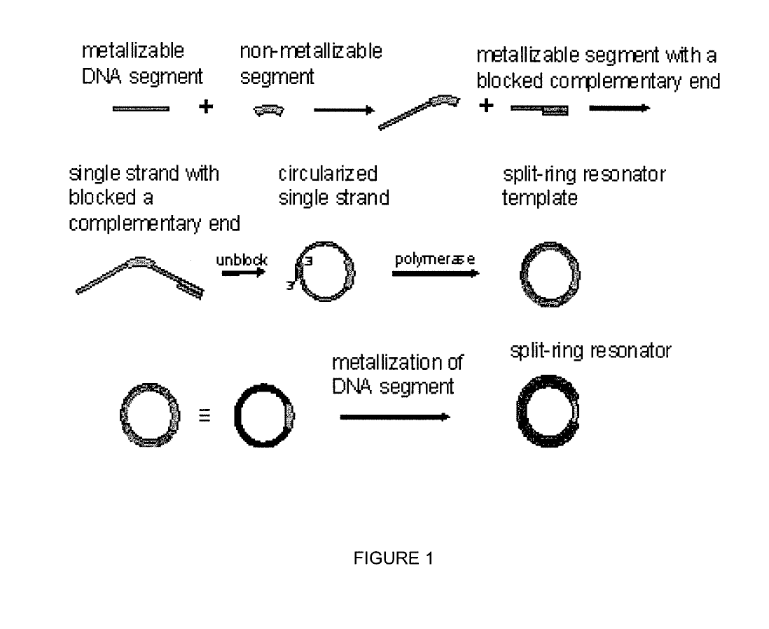

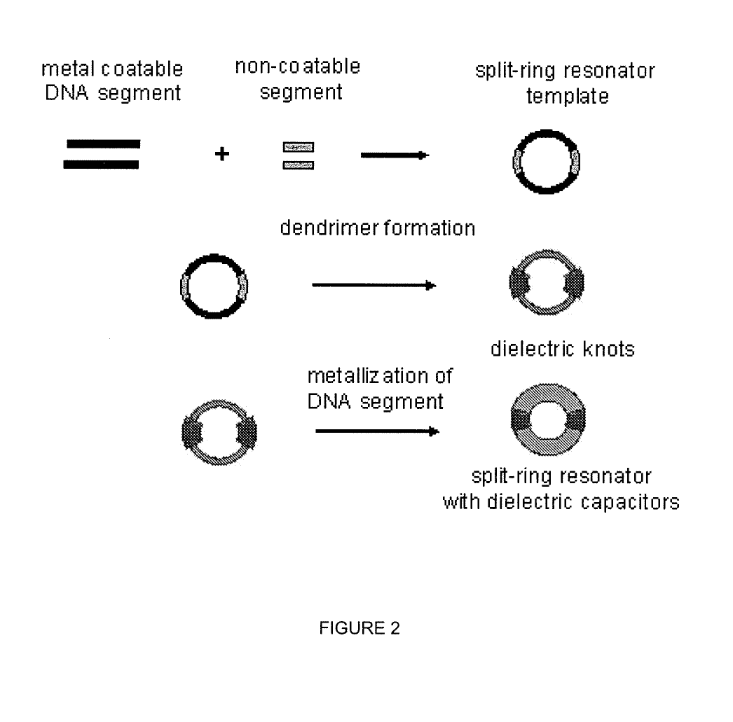

(i) Circuit definition. The first step is the formation of a synthetic single-stranded or doublestranded DNA (or RNA) ring containing one or several tailored molecular spacers, which determine the subsequent location of the unmetallized segments. The ring is prepared by one of the methods described below using standard procedures based on specific base-pair recognition. (Caruthers, M. H. (1982) in Chemical and Enzymatic Synthesis of Gene Fragments, eds. Gassen, H. G. & Land, J. A. (Verlag Chem, Weinheim, FDR), 71-93; Caruthers, M. H. “Gene Synthesis Machines: DNA Chemistry and Its Uses” Science 1985, 230, 281-285; Caruthers, M. H. “Chemical Synthesis of DNA and DNA Analogues” Acc. Chem. Res. 1991, 24, 278-284). FIG. 1 shows the general synthesis of split-ring resonators based on small double-stranded DNA (dsDNA) circles with unmetallized segments. Although dsDNA is relatively stiff with a persistence length of 50 nm, it can be bent, under some conditions, into surprisingly tight loops (r<6 nm) and offers, as a double stranded moiety, the most options for metallization. (Richter, J. “Metallization of DNA” Physica E 2003, 16, 157-173; Shore et al. “DNA flexibility studied by covalent closure of short fragments into circles” Proc. Natl. Acad. Sci. USA 1981, 78, 4833-4837; Ulanovsky et al. “Curved DNA: Design, synthesis, and circularization” Proc. Natl. Acad. Sci. USA 1986, 83, 862-866; Amzallag et al. “3D reconstruction and comparison of shapes of DNA minicircles observed by cryo-electron microscopy” Nucleic Acids Res. 2006, 34, e125-e132). The j-factors for the direct formation of small rings with nm-sized diameters from dsDNA indicate that yields can be expected to be very poor. Most of the methods use a synthesized single stranded DNA (ssDNA) with incorporated unmetallizable segments and complementary ends which are designed to join into a ring by linking with other complementary DNA tail segments. The ring is then metallized by a choice of methods which depend on the type of unmetallizable segment and whether the DNA has been converted to a duplex form. It is anticipated that more abundant and less expensive natural or biosynthetic sources may be available for use, in part, for the formation of the ring templates and several methods are described below that use these potential sources.

(ii) Metallization. The DNA in the ring, but not the hydrophobic organic spacer or possibly the inert DNA spacer, is then metallized by art-known methods appropriate for the specific ring template. (Eichen et al. Acta. Polym. 1998, 49, 663-670; Braun et al. Nature 1998, 391, 775-777; Richter, J. “Metallization of DNA” Physica E 2003, 16, 157-173).

Non-selective metalization of DNA with good electrical conductivity is obtained by treatment with aqueous AgNO₃ or by other known metallization procedures. (Monson, C. F.; Woolley, A. T. Nanoletters 2003, 3, 359-363). The simplest method using AgNO₃ proceeds by ion exchange, which produces nucleation sites for electroless metal deposition. Reaction conditions can be found in several studies. The procedure is non-selective, occurring along the entire length of the dsDNA.

Several methods for making dsDNA metallization selective are well known in the art. (Patolsky et al. “Au-Nanoparticle Nanowires Based on DNA and Polylysine Templates” Angew. Chem. Int. Ed. 2002, 19, 2323-2327; Weizmann et al. “Telomerase-Generated Templates for the Growing of Metal Nanowires” Nano Letters 2004, 4, 787-792). One method chemically bonds a reductant to specific DNA sites. (Keren et al. “Patterned DNA Metallization by Sequence Specific Localization of a Reducing Agent” Nano Letters 2004, 4, 323-326; Keren et al. “Sequence-Specific Molecular Lithography on Single DNA Molecules” Science 2002, 297, 72-75). The part of the ring with the reductant becomes metallized whereas the part without the reductant is inert and remains bare. Other known metallization processes introduce tethered metal nanoparticles at functionalized nucleosides, seed metallization through photochemical attachment of the tethering points directly to a specific nucleoside base (thymine) or through the telomerization of a tether-containing amino oligonucleotide. (Monson, C. F.; Woolley, A. T. Nanoletters 2003, 3, 359-363). Unmetallized segments occur where these modifications are absent.

(iii) Insulation. At high concentrations the metallized rings may touch one another when electrically shorted which deleteriously alters their response to the optical field. To avoid this, the metallized rings are coated with an insulating layer consisting of a single to one or more monolayers. The surface of the silver is insulated by coating with a self-assembled monolayer (SAM), for instance, such as an alkanethiol or a composition comprising an alkanethiol. If needed, this can be functionalized on the outside with initiating sites for the formation of an encapsulating polymer shell to complete the growth of the desired puck (the encapsulated metallized ring). Similar electrical insulation with a monolayer is applied to all other possibly present constituents of the bulk material.

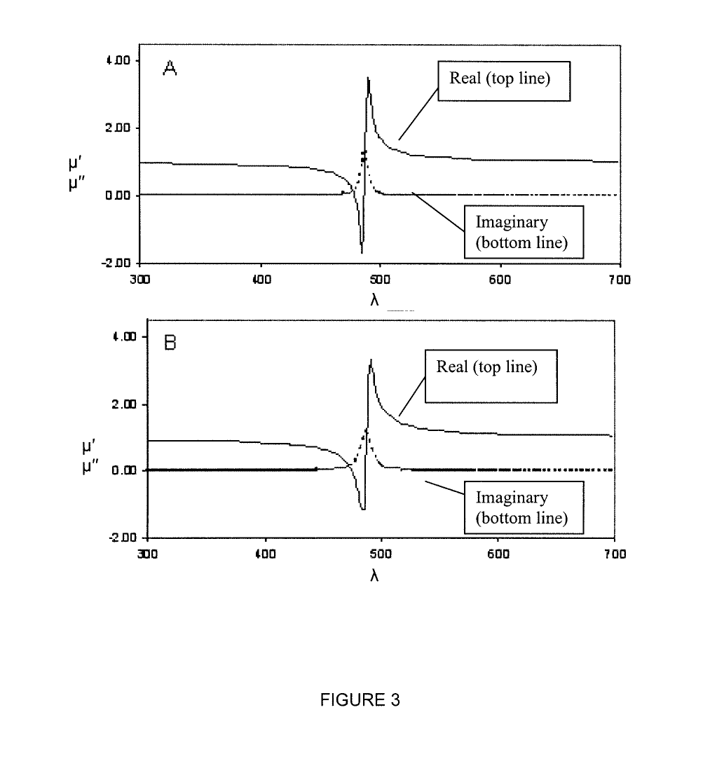

Feasibility and Potential Performance. FIG. 3 shows the real (μ′) and imaginary (μ″) parts of the permeability for a two-break Ag structure as a function of frequency for neat and fivefold diluted isotropic samples, calculated using Maxwell's equations and known properties of silver metal. Control of ε is much easier than that of μ and its value is made strongly negative by use of a mixture of metal particles of appropriate size and shape or an organic dye, if needed. The ring itself is expected to intrinsically have the desired response to the electric field, but if it does not, a two component mixture can be used—one component for controlling ε and one component for controlling μ.

The calculations represented in FIG. 3 are very similar to those of O'Brian and Pendry but include a treatment of the distributed capacitance, as determined by antenna loop theory, for a more realistic result. (O'Brien, S.; Pendry, J. B. “Magnetic activity at infrared frequencies in structured metallic photonic crystals” J. Phys.: Condens. Matter 2002, 14, 6383-6394; O'Brien et al. “Near infrared photonic band gaps and nonlinear effects in negative magnetic metamaterials” Phys. Rev. B 2004, 69, 241101-241105; Kanda, M. “Standard Antennas for Electromagnetic Interference Measurements and Methods to Calibrate Them” IEEE Trans. on Electromagnetic Compatibility 1994, 36, 261-273). The frequency dependence of the conductivity of Ag is accounted for by means of Drude's equation,

$$σ = \frac{{σ}_{0}}{1 - iωσ} = \frac{{ω}_{p}^{2}}{4 {πω}^{2} τ} + i \frac{{ω}_{p}^{2}}{4 πω}$$

where σ₀=ωₚ²T/4 TT, and ωp=1.4×10¹⁶ s⁻¹ and T=3.1×10⁻¹⁴ s are the value bulk silver. (Ishikawa et al. “Negative permeability of split ring resonator in the optical frequency region” Proceedings of SPIE 2005, Vol. 5927).

Adding the distributed capacitance is done to insure that the frequency response for a selected ring diameter is not overly optimistic. The greater cross-ring capacitance requires a smaller inductance to keep the response in the visible, which leads to two possible negative effects: a decrease in the intensity of the response which drops with the geometric inductance and is proportional to r² (r=radius), and frequency saturation which occurs when the geometric inductance falls below the intrinsic inductance of the metal and no further decrease in r can increase the response frequency.

Considering the small dimensions of the “wires” compared with the mean free path of 40 nm for electrons in bulk silver, the use of the bulk value for T may be problematic. This issue has been briefly examined by Panina et al. who have concluded that effects on the real conductivity will be small when ωT>>1. (Panina et al. “Optomagnetic composite medium with conducting nanoelements” Phys. Rev. B 2002, 66, 155411-155428). This condition might not be met if the small dimensions and grain sizes comparable to the mean free path reduce T sufficiently. To assess the potential effect due to a decrease in T, the ad hoc assumption was made that T is reduced by a factor given by the device wire radius divided by the mean free path (8 nm/40 nm=0.2). Under this very stringent restriction, more severe than the previously used adjustments of T, the calculation no longer predicts a negative real part μ′. (Linden et al. Science 2004, 306,1351-1353; Zhang et al. Phys. Rev. Lett. 2005, 95, 137404-137408). However, as shown below, even then success is still achievable.

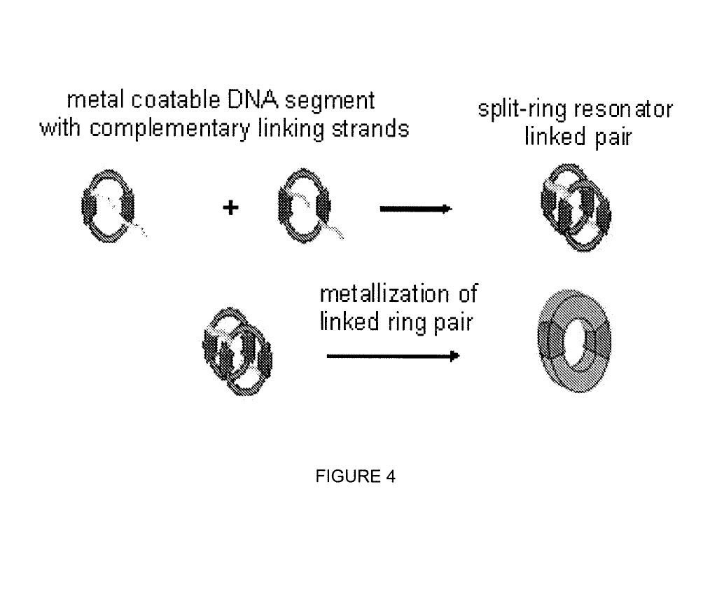

There are two options for addressing the potential problem directly: (i) finding a better conducting metal or (ii) adding more metal to the active device. It is believed silver has the best intrinsic properties at room temperature and this leaves only option (ii). Fortunately, adding metal without increasing the ring radius so as to maintain λ>>2 r is certainly possible by increasing the solenoid character of the ring through an addition to the metal thickness parallel to the axis of the ring. This makes the ring inductance larger and simultaneously moves T in the direction of the bulk value. A set of self-assembled, stacked DNA rings (FIG. 4) is a straightforward extension of the synthetic method proposed herein. Each ring is designed to have several complementing pigtails that match between rings to form a stack, which when metallized form a thick cut ring with a flat aspect ratio to minimize the increase in the geometric inductance.

Recently measured resistivities of metallized DNA cover a wide range of numbers from seven times the bulk value on 75 nm Au wires to values that are several orders of magnitude greater than the bulk value. (Keren et al. Science 2002, 297, 72-75; Liu et al. “DNA nanotube self-assembled from triple-crossover tiles as templates for conductive nanowires” Proc. Nat. Acad. Sci. 2004, 101, 717-722; Ongaro et al. “DNA-Templated Assembly of Conducting Gold Nanowires between Gold Electrodes on a Silicon Oxide Substrate” Chem. Mater. 2005, 17, 1959-1964). They appear to depend greatly on whether the wires are overlaid by the electrodes or vice versa and on wire thickness, granularity and uniformity. (Harnack et al. “Tris(hydroxymethyl)phosphine Capped Gold Particles Templated by DNA as Nanowire Precursors” J. M. Nano Lett. 2002, 2, 919-923). All are lower than those first reported by Braun (Nature 1998, 391, 775-777). High resistivity of the rings dampens the resonance response but also may be controllable by thickening the metal along the ring axis. Effort to improve the single-ring response is preferable in order to maintain high dilutions within the bulk, and great attention is preferably paid to optimizing the metallization conditions. High dilution helps reduce undesirable, anti-ferromagnetic, inter-ring inductances that can reduce the response, while simultaneously making the qualities of the bulk resemble the diluent, which can be separately optimized for the specific device.

The distribution of the critical geometric ring parameters such as diameter, gap distance and metal thickness and uniformity are preferably as narrow as possible to prevent deleterious broadening of the resonant response.

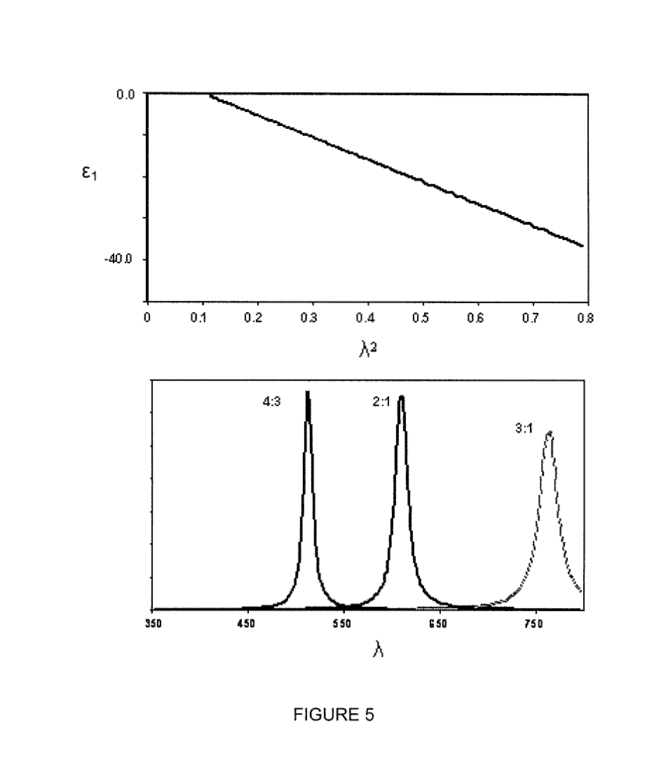

Negative permittivity of bulk materials. The real part of the permittivity (ε=ε′+iε″) is negative for silver throughout the optical frequency range (FIG. 5 top) insuring that the condition that both εμ<0 will be satisfied for any μ(λ)<0. The imaginary part of ε can become quite large due to a plasmon resonance that occurs in small metal nanoparticles with dimensions much smaller than λ. For such a small spherical metal nanoparticle the ratio Q of the absorption cross section to the geometrical cross to the geometrical cross section ar² is given by,

$$Q = ( 2 π a {ɛ}_{0}^{1 / 2} / λ ) Im ( \frac{ɛ - {ɛ}_{0}}{[ ( ɛ + {χɛ}_{0} ) ]} )$$

where X (upper case chi) is a geometrical correction factor and is equal to two for a sphere and ε₀ is the dielectric constant of the medium the particles are embedded. The plasmon resonance has a maximum when Re(ε(λ))=−Xε₀. (Kelly et al. “The Optical Properties of Metal Nanoparticles: The Influence of Size, Shape, and Dielectric Environment” J. Phys. Chem. 2003, 107, 668-677; Zeman et al. “An Accurate Electromagnetic Theory Study of Surface Enhancement Factors for Ag, Au, Li, Na, Al, Ga, In, Zn, and Cd” J. Phys. Chem. 1987, 91, 634-643). For less symmetric shapes, X is larger than two leading to a red shifted resonance to that of a sphere. The above relationship is used to estimate the wavelength of the plasmon resonance a metal ring with different metal thicknesses. Noting that X increases as the radius to metal thickness ratio increases, FIG. 5 (bottom) qualitatively shows the variation of the resonance with this ratio, neglecting radiative dampening and depolarization effects which broaden the resonance. FIG. 5 also demonstrates how the high opacity of the resonance region can be avoided by adjusting the metal thickness which only slightly changes the magnetic resonance.

Other Materials. The synthesis of materials with μ<0 allows continuous control of both the magnitude and the sign of the real and imaginary parts of μ. The methods described here are available for use in other materials with tailored refractive indices. An example is a material with a zero real refractive index.

Measurement. The refractive index of the optical LHM can be deduced from the thin film reflectance and transmission spectra following standard methods of analysis, including curve fitting and simulation where appropriate, to obtain the dispersion curves of the complex ε and μ. (Shalaev et al. Opt. Lett. 2005, 30, 3356-3358; Linden et al. Science 2004, 306, 1351-1353; Zhang et al. Phys. Rev. Lett. 2005, 95,137404-137408; Grigorenko et al. Nature, 2005, 438, 335-338).

### Methods: Common Practices, Nomenclature and Conventions.

The DNA length is determined by taking the desired outer radius (R_{od}) after metallization and subtracting half of the desired metal thickness (Tₘₑₜₐₗ). The full circumference is calculated from this center radius and reduced by the required gap width L_{gap} to obtain the final length, C_{DNA}=[2TT(R_{od}−Tₘₑₜₐₗ/2)-L_{gap}]. The number of base pairs (bp) is found by dividing C_{DNA} by 0.33 nm/bp. For the target device of FIG. 3: R_{od}=40 nm, C_{DNA}=181 nm and bpₜₒₜₐₗ=548.

For a typical method, multiple ssDNA oligomers are synthesized with non-complementary and complementary base sequences that are not explicitly given in this disclosure but are assumed to be chosen to optimize duplex formation and minimize strain and preferably is approximately 60% guanine-cystine (G-C) bp to maintain a high melting point. (Levine et al. “The Relationship of Structure to the Effectiveness of Denaturing Agents for Deoxyribonucleic Acid” Biochem. 1963, 2, 168-175). Strain in these very small circles can be relieved by including flexible TATA boxes, a specific sequence of bases that promotes bending in duplex DNA. (Davis et al. “TATA Box Deformation with and without the TATA Box-binding Protein” J. Mol. Biol. 1999, 291, 294-265). The base length of a oligomer is L_{DNA}=C_{DNA}/N−L gap, where N is the number of gaps, and bp*=L_{DNA}/0.33 nm/bp, rounded to the nearest integer.

The inert and unmetallizable molecular spacers (S) of the ring template are the capacitive components of the active device and are designated, (A)-5′-S-3′-(B), to indicate how the ends of the ssDNA oligomers, A and B, are attached. Two types of molecular spacers can be used: hydrophobic organic rigid rod and inert duplex DNA. The hydrophobic kind can be straight or bent rigid rods consisting of an organic structure with end groups, such as carboxylic, acids that can be easily attached to DNA. (Schwab et al. “Molecular Rods. 1. Simple Axial Rods”, Chem. Rev. 1999, 99, 1863; Schwab et al. “Synthesis and Properties of Molecular Rods. 2. Zig-Zag Rods,” Chem. Rev. 2005, 105, 1197). For the example of a carboxylic acid this is done through an ordinary peptide bond coupling to 5′-amino terminated DNA. In the methods below, oligo(phenylene acetylenes) were chosen whose dicarboxylic acids in lengths up to 12.4 nm are available in good yields, as a preferred, but not required, example of a hydrophobic rigid-rod spacer molecule. (Tour, J. M. “Conjugated Macromolecules of Precise Length and Constitution. Organic Synthesis for the Construction of Nanoarchitectures” Chem. Rev. 1996, 96, 537-553). Others that may be very useful are the thicker poly(pentiptycenes). (Yang et al. “Probing the Intrachain and Interchain Effects on the Fluorescence Behavior of Pentiptycene-Derived Oligo(p-phenyleneethynylene)s” J. Am. Chem. Soc. 2006, 128, 14109-14119; Yang, J.-S.; Ko, C.-W. “Pentiptycene Building Blocks Derived from Pentiptycene Quinones” J. Org. Chem. 2006, 71, 844-847). They are also rigid with well defined lengths but have a larger radius in the axial direction which is very useful for preventing encroachment of the metallization into the gap. The dicarboxylic acid derivatives of this class are also easily synthesized. Inert DNA spacers are based on the inclusion of derivatized bases into the ring template in a manner that allows gaps to be made by selective metallization.

All exemplary methods described require some automated DNA synthesis on a solid support (ss). Removal from and attachment to the solid support are done by standard methods. (Caruthers, M. H. Science 1985, 230, 281-285; Caruthers, M. H. Acc. Chem. Res. 1991, 24, 278-284).

Various abbreviations and conventions are used in the method sections and figures below: base pair (bp), inert spacer (S), solid support (ss), single-stranded DNA (ssDNA), duplex or doublestranded DNA (dsDNA), non-complementary (U), complementary base pair sequence (Uu′, Yy, Zz, etc.), the pertinent conditions and detail of a repeated reaction (reaction letter) is given upon its first use. Methods use the following key: (A) organic rigid rod molecular spacers; (B) inert molecular spacers made from dsDNA; (C) ring templates made of only ssDNA; (D) dendrimerization; (E) telomer complexed ssDNA rings; (F) stacked rings; (1) single gap; (2) multiple gaps; (a) molecular spacer conjoined 5′-S-5′; (b) molecular spacer conjoined 5′-S-3′; (c) molecular spacers conjoined by restriction enzyme overhangs.

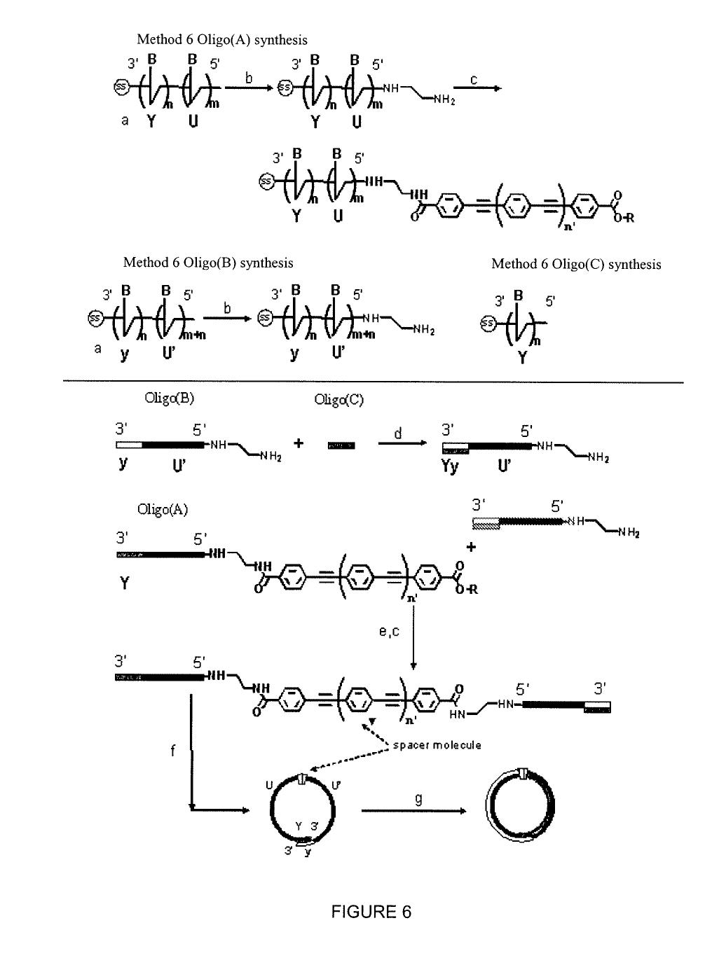

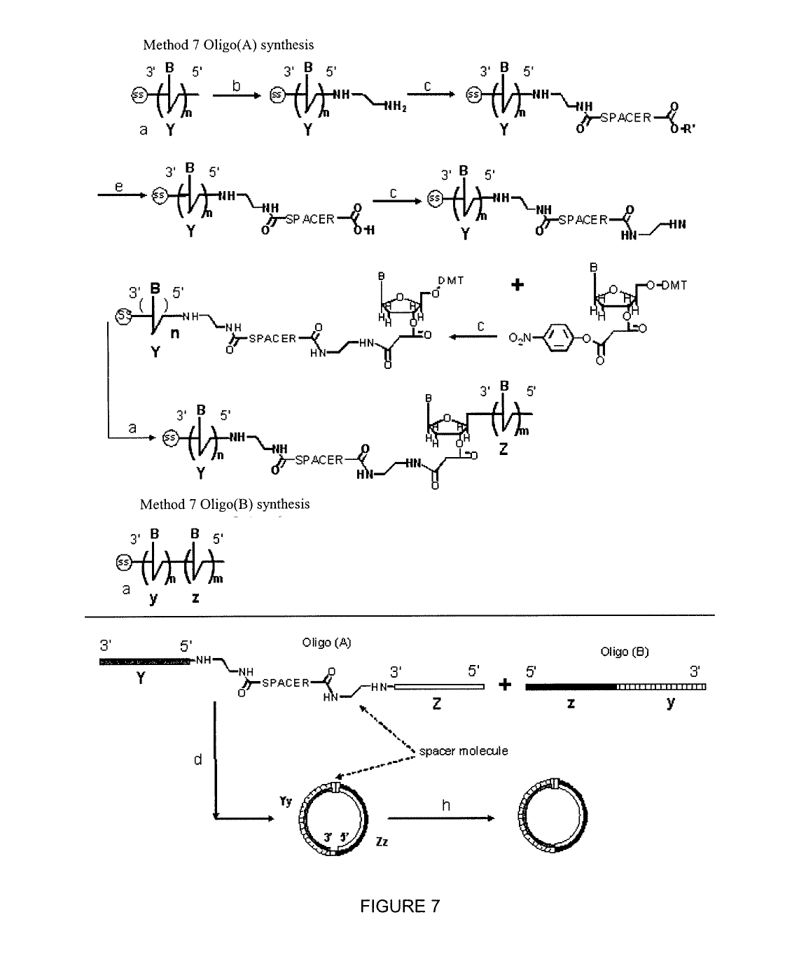

For convenience, the methods described below are labeled according to the corresponding figures, for example, the method illustrated in FIG. 6 is labeled Method 6, the method illustrated in FIG. 7 is labeled Method 7, and so on.

Method 6: dsDNA Ring Template with a 5′-S-5′ Coupling of a Single Inert Organic Spacer.

A dsDNA ring template is synthesized by joining two oligomers, A and B, at their 5′-ends to an inert organic spacer molecule and then closing the circle by allowing the unconnected and complementary 3′-ends to overlap and form a duplex DNA link (FIG. 6). Oligomer A is made with a specified sequence of bp in the usual 3′ to 5′ direction by standard automatic synthetic methods and is comprised of two parts: A short sequence (Y) of n bp that starts at its 3′-end and is specified to be complementary to an equal length sequence on oligomer B; and a long, non-complementary sequence (U) of m bp, such that bp*=m+n which brings the ring to its required circumference when completed. The length n is expected to be 18-25 bp since the overlapping segments acts as the primer for the DNA polymerase. Ethylene diamine is attached to the 5′-end of the oligomer while it is still attached to the solid support by standard methods (reaction b). (Chu et al. “Derivatization of unprotected polynucleotides” Nucl. Acids Res. 1983, 11, 6513-6529; Chollet et al. “Biotin-labled synthetic oligodeoxyribonucleotides; chemical synthesis and uses as hybridization probes” Nucl. Acids Res. 1985,13, 1529-1541). An inert organic spacer molecule with carboxylic acid groups at each end is prepared beforehand for joining oligomers A and B by protecting one of its carboxylic acid groups by esterification. The spacer molecule is attached through a peptide bond made to its unprotected carboxylic acid group (reaction c). The gap length of 7.2 nm corresponds to using n′+1=8 poly(phenylethynylene) oligomer units and allowing for a 1.3 nm adjustment for the two connecting groups.

Oligomer B is also autosynthesized, but its non-complementary segment (U′) is lengthened by n bp to compensate for the linking overlap. Bringing the total circumferential number of bp to 2×(m+n)=548. A n-bp sequence (y) that is complementary to the n-bp sequence (Y) of oligomer A initiates the 3′ end. At the completion of the synthesis, the 5′-end is amino terminated (reaction b).

Oligomer C is n-bp long and matches the n-bp sequence (Y) on oligomer A and is combined with oligomer B by (reaction d). The function of oligomer C is to block the undesired linking of the 3′-ends of oligomers A and B during subsequent steps.

The remaining carboxylic acid group attached to the molecular spacer segment of oligomer A is deprotected (reaction e) and coupled, through peptide bond formation (reaction c), to the protected oligomer B to make the 5′-S-5′ conjoined oligomer AB. Finally, the ring is closed by denaturing the conjoined oligomer AB by heat and slow cooling to in dilute solution (reaction f). Under these conditions the ring is formed preferentially over chains. (Shore et al. “DNA flexibility studied by covalent closure of short fragments into circles” Proc. Natl. Acad. Sci. USA 1981, 78, 4833-4837). The mostly ssDNA circle is now converted to a nearly complete duplex circle by application of polymerase (reaction g) e.g., Taq DNA polymerase.

Method 7: dsDNA Ring Template with a 5′-S-3′ Coupling of a Single Inert Organic Spacer.

As illustrated in FIG. 7, oligomer (A) begins with n-bp specified sequence (Y) made in the usual 3′ to 5′ direction on a solid support by standard automated methods, with n=bp*=274 for R_{od}=40 nm and 5′-amino terminated by reaction b. Its 5′ end is coupled to a protected spacer molecule by reaction c. Deprotection (reaction e) followed by formation of a peptide bond (reaction c) with ethylene diamine (or other diaminoalkane) produces a 5′-S-NH2 moiety that is used to restart the automated DNA 3′ to 5′ synthesis after a primer oligonucleotide containing a 3′-p-nitrophenylsuccinate ester is attached by a second peptide bond (reaction c). The automated synthesis is continued through a second specific sequence (Z) until n-1 bp are added. The length of the organic spacer is adjusted to compensate for the presence of the primer nucleoside so that the correct length of the unmetallized portion of the ring is obtained.

Oligomer B is a double set of specified complementary bp sequences (y, z) made by standard methods (reaction a). Its 3′ to 5′ sequences (y,z) are chosen to be complementary to 5′ to 3′ sequences (Y,Z) of oligomer A so that when the two are combined and heated (reaction d) and cooled, a dsDNA circle is formed. Treatment with ligase (reaction h) closes the gap made by the 3′ and 5′ ends of oligomer A. (Ulanovsky et al. Proc. Natl. Acad. Sci. USA 1986, 83, 862-866).

Method 8: dsDNA Ring Template with a Single 5′-S-5′ (or 5′-S-3′) Coupled Inert Organic Spacer Joining a Single Duplex DNA Strand.

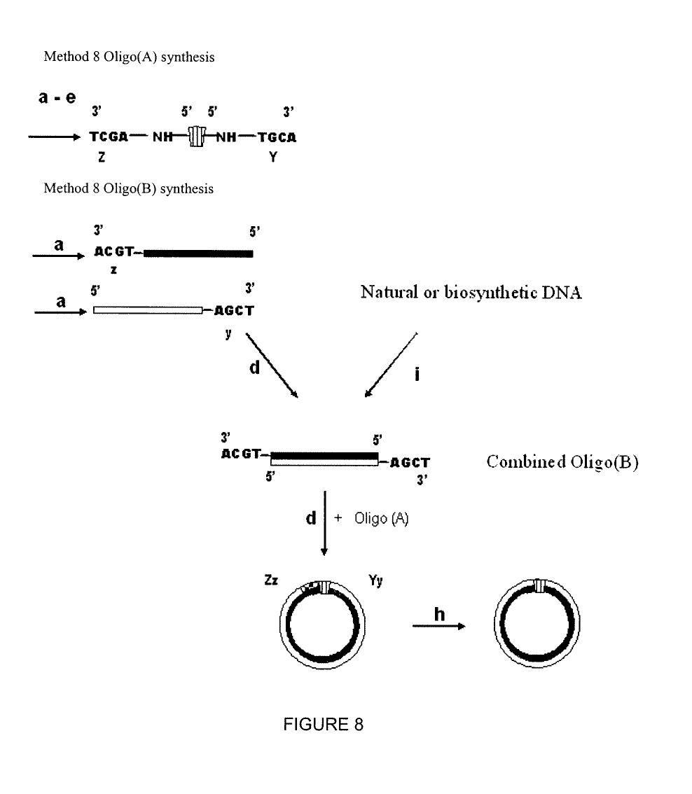

This method (FIG. 8) recognizes the use of inexpensive and more abundant natural or biosynthetic sources of duplex DNA that can be processed by the action of restriction or other enzyme types into dsDNA of the correct length for the formation of ring templates. To utilize this source of DNA, the method takes a pair of short (at least four bp) ssDNA oligomers joined 5′-S-5′ by Method 6 and uses this conjoined oligomer to link the duplex strand of enzyme-processed natural or biosynthetic DNA (548 bp, Rod=40 nm) into a circle with a single inert organic spacer. The conjoined oligomer is shown in FIG. 8 as oligomer A and has two specified 3′-terminated 4-bp sequences coupled to an organic 5′-S-5′ inert spacer molecule by Method 6. The terminating sequences of oligomer A are synthesized to be complementary to the 3′-terminal overhangs of the duplex DNA (oligomer B) that remain after the action (reaction i) of two different restriction enzymes, e.g., Pstl (providencia stuartii) Sacl and (streptomyces achromogenes). (Lodish, H. F. “Molecular Cell Biology” 5th ed., W.H Freeman and Co.: New York, 2003). Oligomer B is anticipated to be produced from a naturally occurring larger piece of duplex DNA by the application of a pair of restriction enzymes (reaction i) and can also be synthesized by standard automated methods (reaction a), if necessary. The circle is completed by the combination of oligomers A and B at low temperature (reaction d) at dilute concentrations to maximize ring formation over chains and the final application of ligase (reaction h).

For the most general application of this method, it is assumed that there is facile rotation of the duplex DNA about the bonds of the organic spacer such that the number of twists of the duplex DNA around the circle does not have to be an integer number and that any circumference within the resolution limited by a bp is possible.

The method is easily adaptable to 5′-S-3′ inert spacer molecule by altering the set of restriction enzymes from a pair that makes two different 3′-terminal overhangs to a mixed pair containing one enzyme that makes a 5′-terminal overhang. In all cases the specific terminal 3′- or 5′-bp sequences of the conjoined oligomer A are determined by the choice of restriction enzymes.

To form a dsDNA ring template with multiple 5′-S-5′ (or 5′-S-3′) coupled single inert organic spacers doing multiple duplex DNA strands, a method analogous to Method 8 can be used except that a set of 2N different restriction enzymes are applied to the natural or biosynthetic DNA and an equivalent set of Yn-5′-S-5′(or 3′)-Zn-1 conjoined oligomers.

Method 9: dsDNA Ring Template with 5′-S-5′ Couplings of Multiple Inert Organic Spacers.

Ring templates that lead to smaller capacitances than those obtained from a single gap are necessary for a very high material response frequency. Very small capacitances are obtained by adding multiple gaps to a single ring, where Cring=Cgap/N, and Cgap and N are the single-gap capacitance and the number of gaps included in the ring.

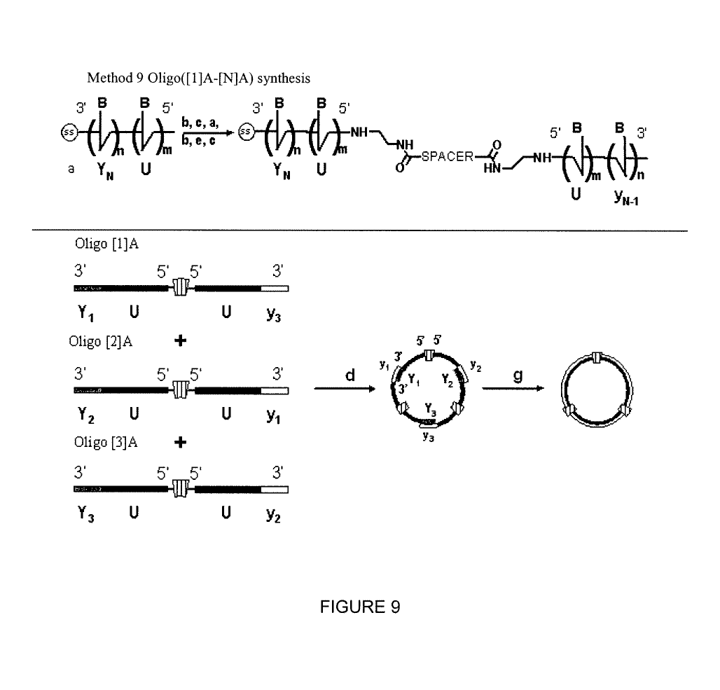

To synthesize a ring template with N gaps, N ssDNA oligomers, [N.A. with Yn-5′-S-5′-yn-1 coupled complementary sequences (Yn, yn+1)], are prepared by Method 6 except that the n-bp complementary sequences at the 3′ ends are now used to cross link to oligomers [n−1]A and [n+1]A in a manner that leads to the formation of a complete loop. These oligomers cannot link internally and make the synthesis of oligomer C of Method 6 unnecessary. The basic length of the DNA strand is given by the formula for LDNA given above with bp*=m+n with n sufficiently long to act as a polymerase primer. The method is illustrated for a ring with three gaps in FIG. 9. Three 5′-S-3′ oligomers, 1A, 2A and 3A, are brought together in dilution at low temperature (reaction d) so that their complementary tail sequences (Y1 ,Y2,Y3,y1,y2,y3) allow the 3′ ends to daisy chain and form a complete circle. The template is converted to duplex form by polymerase (reaction g).

Method 10: dsDNA Ring Template with 5′-S-3′ Couplings of Multiple Inert Organic Spacers.

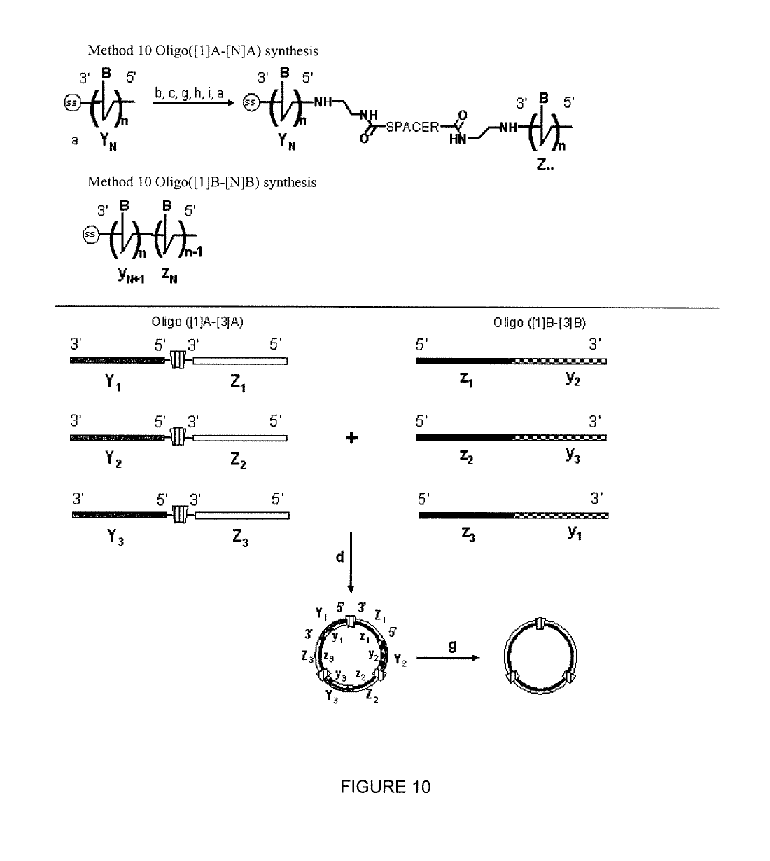

Multiple 5′-S-3′ inert spacer molecules can be incorporated in a manner similar to Method 9. The set of N A-type oligomers, Yn-5′-S-3′-Zn-1 is synthesized by Method 7. The complementary 3′ to 5′ bp sequences (Yn, Zn) of oligomers A permit a daisy chain assembly of a full circle when they are combined with the set of N B-type oligomers with complementary sequences (yn+1, Zn), as shown in FIG. 10. Ligase (reaction h) is applied to join the ends of the N.A.-type oligomers in the duplex ring.

Method 11: dsDNA Ring Template with One or More Inert dsDNA Segments.

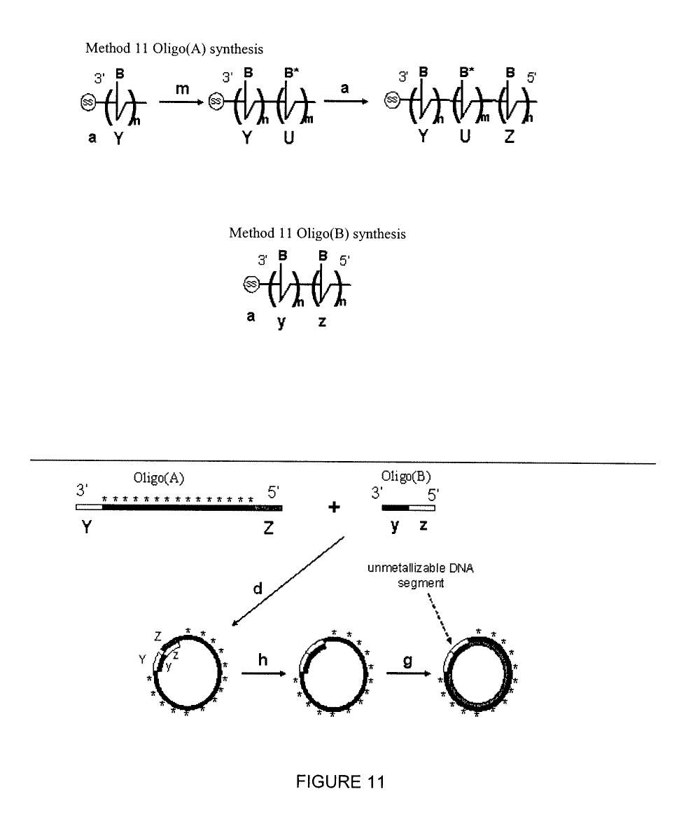

A ring template is constructed by this method entirely from DNA with no organic spacer molecule(s) (FIG. 11). Part of the ring is synthesized from nucleosides derivatized to promote selective metallization through one of the methods referred to above. The nucleoside derivatives can include premodified bases that include a reducing reagent such as glutaraldehyde or those that have been modified to have doorways for easy functionalization after they have been incorporated into an oligonucleotide. (Tarn et al. “Synthesis and Characterization of Fluorenone-, Anthraquinone-, and Phenotiazine-Labeled Oligodeoxynucleotides: 5′-Probes for DNA Redox Chemistry” J. Org. Chem. 2000, 65, 5355-5359; Burley et al. “Directed DNA Metallization” J. Am. Chem. Soc. 2006, 128,1398-1399; Khan et al. “Palladium(0)-Catalyzed Modification of Oligonnucleotides during Automated Solid-Phase Synthesis” J. Am. Chem. Soc. 1999, 121, 4704-4705).

The formation of the ring follows Method 7 and begins with the synthesis of two oligomers, A and B. Oligomer A is much longer with 2n+m bp (m=548, R_{od}=40 nm), where n is the length of the terminal complementary sequences (Y,Z) and determined by n=L_{gap}/0.33 bp/nm=22 (L_{gap}=7.2 nm). The inert region of the ring is determined by the 2n-bp sequence of underivatized bases that is used to close the circle. Oligomer B has two different n-bp sequences (y,z) complementary to those of oligomer A. The middle m-bp sequence (U) in oligomer A is synthesized from derivatized bases B* (reaction m) and is the crucial element of this method. The final duplex ring is formed by complexing oligomers A and B (reaction d), followed by ligase treatment (reaction h) and duplex formation by polymerase (reaction g). The polymerase must be chosen to work on the oligomers with derivatized nucleosides and is primed by the 2n-bp inert sequence. The Pwo DNA polymerase has been found to work well with derivatized bases. (Burley et al. “Directed DNA Metallization” J. Am. Chem. Soc. 2006, 128,1398-1399). If an inert DNA sequence shorter than the primer length is required and adding derivatized bases to the ends of oligomer B will be necessary. The finished ring consists of two regions: a 2n-bp inert segment that becomes the unmetallized gap and a derivatized m-bp segment that is primed for selective metallization. The above procedure is extendable to connecting N segments in the daisychain manner of Method 10, if they have the appropriately designed complementary units, into a ring template with N gaps.

Method 12: dsDNA Ring Template Synthesized from One or More Inert Duplex DNA Segments and One or More Active Duplex DNA Segments.

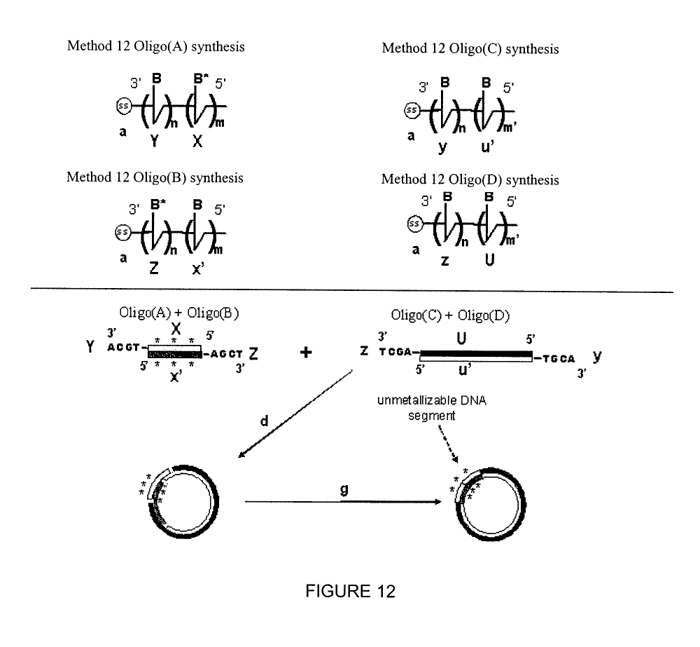

This method (FIG. 12) uses duplex DNA from natural or biosynthetic sources. DNA from this supply is metallizable using the non-selective methods described above if the short dsDNA segments defining the gap regions are derivatized in a manner that blocks metalization. This can be accomplished by several methods: (i) altering the DNA backbone to exclude exchangeable metal ions (e.g. make it neutral), (ii) attaching an enveloping barrier (e.g. dendrimerization through method 15) or (iii) blocking it with an enzyme or protein complex.

Two duplex DNA strands are required. The first strand must be synthesized from two designed oligomers A and B, each having a linkable tail and a derivatized m-bp sequence following Method 11 (reactions a and m). The complementary m-bp sequences (X,x′) of oligomers A and B (m=22, gap=7.2 nm) must be synthesized with derivatized bases that do not interfere with their complementarity. They have a total of n+m bp and the shorter n-bp sequences are complementary to the overhangs of two selected restriction enzymes. Oligomers A and B are combined (reaction d) to make a short (2n+m)-bp dsDNA strand with a pair of 3′-terminal overhangs (5′-terminal or mixed overhangs can also be used). The second duplex DNA strand with m′-bp and two 3′-terminating overhangs is generated by restriction enzymes working on a larger strand of duplex DNA obtained form a natural or biosynthetic source or using autosynthesis following the above methods. The total length is m+m′ bp pairs (m+m′=548 bp, R_{od}=40 nm). Combination of the two duplex strands through their complementary overhangs (reaction d) and ligase application (reaction h) completes the ring template. If a natural or biosynthetic source is impractical, direct synthesis of the second strand is outlined in FIG. 12. Ring templates with multiple gaps can be synthesized by modification of this method.

Method 13: ssDNA Ring Template with Inert DNA Segment.

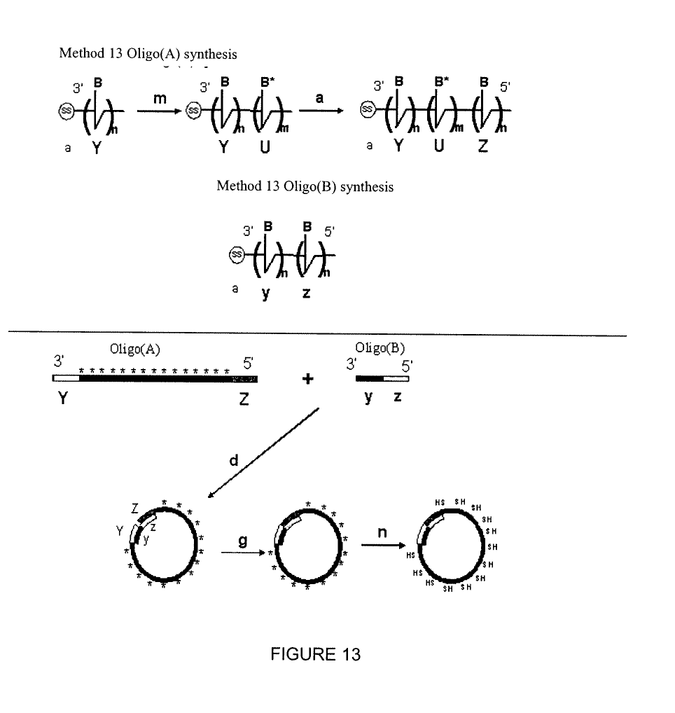

Ring templates of mostly ssDNA have the potential disadvantage of not being a rigid scaffold for the metal but the advantages of a less expensive product and several selective metallization methods. This method follows Method 11 with the following differences: (i) The doorway is to be accessible through the ‘click’ reaction (reaction n) so that thiol groups for selective metalization through nanoparticles can be attached. (Burley et al. J. Am. Chem. Soc. 2006, 128,1398-1399). (ii) The polymerase step is omitted to keep the template single stranded. The derivatized oligomer A (FIG. 13) is combined with the linking oligomer B (reaction d) and followed by ligase (reaction h).

### Method 14: Dendrimerization of the Inert Organic Spacer.

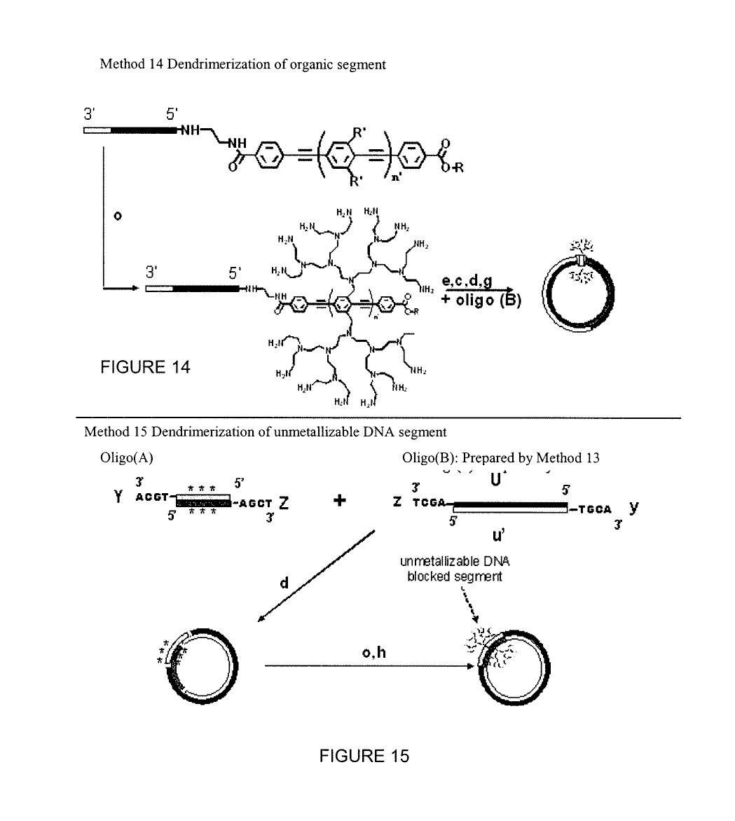

The metallization of the DNA into thick rings may cause a narrowing of the capacitive gap region (FIG. 14). This is easily controlled by starting with a larger gap if the growth rates on the metallization step can be well characterized. A narrow distribution of gap widths for a set of rings is critical for a system with a weakly negative permeability and simple kinetic control may be inadequate. Making the organic spacer thicker is a way of excluding metal growth into the gap. Dendrimer growth along the inert space molecule is an example of a synthetic path for making the spacer thicker. The starburst dendrimer system is one many such a dendrimer systems that can be adapted for this purpose. To grow a dendrimer the spacer molecule is derivatized to have multiple attachment points dendrimer growth initiation (e.g for starburst dendrimers R′ of FIG. 14 can be ethyl amine). The thickness of the dendrimer is controllable by the number of growth cycles applied and lateral control is maintained by limiting the initiation sites to the central portion of the molecular spacer.

### Method 15: Dendrimerization of the Inert DNA Spacer.

This method is used in tandem with other methods such as Method 11 or 12. Dendrimer (e.g. starburst dendrimer) growth is initiated from the derivatized nucleoside doorways placed oligomers A and B, which are subsequently combined to make the inert duplex (FIG. 15). An alternative route is to grow the dendrimer after the derivatized dsDNA system has been incorporated into the completed ring template.

Method 16: dsDNA Ring Templates from Derivatized Telomers with Inert DNA Spacers.

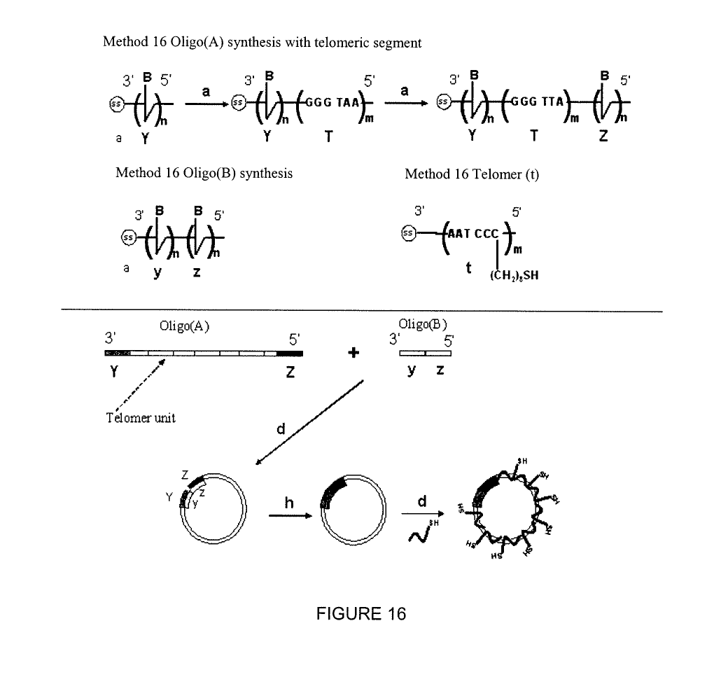

The basic ssDNA ring template of this method (FIG. 16) is synthesized by one of the above methods, such as Method 11, beginning with two ssDNA oligomers A and B, that make a closed circle through one or more complementary overlaps (Yy, Zz). In this method the overlap becomes the inert DNA spacer. Oligomer A is modified from Method 11 to have its middle segment a set of m repeating k-bp telomer sequences such that the total number of bp forming the circle is given by mk with 2n-bp forming the inert DNA spacer section. The telomer segment shown in FIG. 16 has k=6. Automatic synthesis or a natural source of the telomer are both possible. The telomer is derivatized to be a complexation site for a metal cluster. FIG. 16 shows the attachment of octadecylthiol, which binds gold or silver nanoparticles. In the final step (reaction d), the ssDNA ring template is exposed to the telomer which complexes to it and makes a series of evenly spaced (˜2 nm for the k=6) binding sites for metal nanoparticles along the part of the ring to be metallized. The inert DNA spacer is augmented by one telomeric unit.

### Method 17: Stacked Ring Templates.

Good optical response depends on simultaneously keeping both R_{od}<<λ and the resistivity of the metallized ring as small as possible. Without changing the metal, the easiest way to reduce the resistivity is to increase the thickness of the metal, but, unless the metal growth is controlled in some manner, increased thickness leads to a larger R_{od}. Stacking ring templates is a way to control metal growth and lower resistivity without increasing R_{od}. If the rings templates are stacked in solenoidal fashion, with an inter-ring, axial spacing closer than the radial metal thickness, the metalization of the stacked templates span the space between them and lead to a conductive bridge. This effectively makes the metal growth non-isotropic and favored in the axial direction. Multiple rings can be thus stacked to create cut low-resistance cylindrical sheets with an enhanced magnetic response.

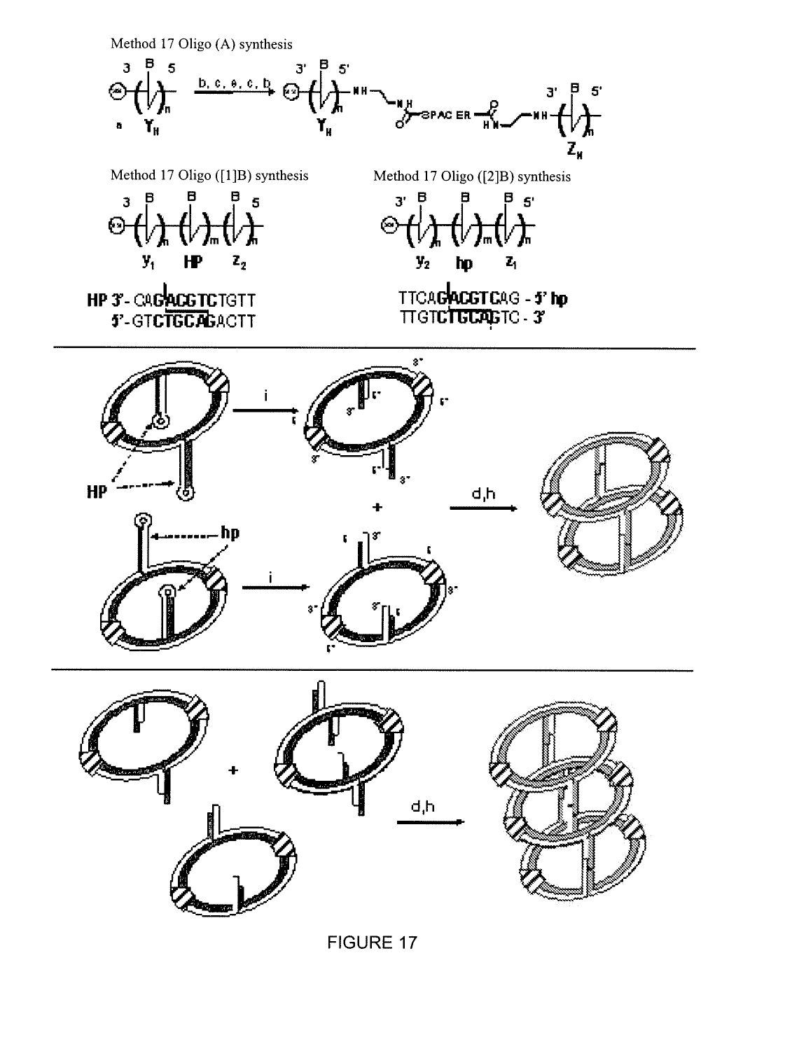

Ring template stacking is possible with all of the dsDNA ring template methods. It is illustrated in FIG. 17 as a modification of Method 10. A duplex DNA ring template with two inert spacers is synthesized exactly by Method 10 except that the oligomer B includes two separate k-bp segments that have a specified bp sequence that produces a duplex hairpin loop. (Xodo et al. “DNA hairpin loops in solution. Correlation between primary structure, thermostability and reactivity with single-stranded-specfic nuclease from mung bean” Nucl. Acids. Res. 1991, 19, 1505-1511). The value of k is determined by the number of required twists and the desired axial stacking separation. One full DNA twist occurs every 10.5 bp and has a length of 3.5 nm. The number of twists is very important when the spacers are made from inert DNA. In this case, if the total number of twists is not integral, highly strained or incomplete templates result with no conformational integrity. However, when organic inert spacers are used, rotation of the DNA about the spacer bonds relax the integral twist number constraint. Using inert DNA segments with free rotation gives better control of the final position of the hairpins relative to the ring plane. The desired conformation is to have both hairpins cis to the plane. Modeling of the ring template provides guidance for obtaining the cis hairpin conformer.

FIG. 17 also shows two double-gap ring templates, each with a pair of hairpin loops. Within each of these hairpins is a unique five-bp sequence that can be recognized by a restriction enzyme and four sequential thiamine bp that make the hairpin bend. One restriction enzyme is used for each inter-ring pair of hairpins. The directionality of the recognition sequences is reversed between the two different templates so that when application of the restriction enzymes is made separately to the top and bottom rings, the enzymes cut complementary overhangs into the hairpins. Combining the two templates after the overhangs have been made results in a stacked pair.

Multiple stacks are also obtainable. FIG. 17 shows a triple set of rings. Multiple stacks are made in the same way except two sets of hairpin pairs are necessary for each non-terminal ring, with a specific enzyme for each set of connecting hairpins.

Substitution of RNA. RNA is easily substituted for DNA in many of the methods and can be used where advantageous.

Insulation with alkanethiols. The surface of the silver is insulated by coating with a selfassembled monolayer (SAM) of an alkanethiol (10-4-10-3 M alkanethiol in ethanol). If needed, this can be functionalized on the outside with initiating sites for the formation of an encapsulating polymer shell to complete the growth of the desired puck. Similar electrical insulation with a monolayer is applied to all other possibly present constituents of our bulk material.

Having now fully described the present invention in some detail by way of illustration and examples for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

One of ordinary skill in the art will appreciate that starting materials, reagents, purification methods, materials, substrates, device elements, analytical methods, mixtures and combinations of components other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

When a group of materials, compositions, components or compounds is disclosed herein, it is understood that all individual members of those groups and all subgroups thereof are disclosed separately. When a Markush group or other grouping is used herein, all individual members of the group and all combinations and subcombinations possible of the group are intended to be individually included in the disclosure. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure. In the disclosure and the claims, “and/or” means additionally or alternatively. Moreover, any use of a term in the singular also encompasses plural forms.

All references cited herein are hereby incorporated by reference in their entirety to the extent that there is no inconsistency with the disclosure of this specification. Some references provided herein are incorporated by reference to provide details concerning sources of starting materials, additional starting materials, additional reagents, additional methods of synthesis, additional methods of analysis, additional biological materials, additional nucleic acids, chemically modified nucleic acids, additional cells, and additional uses of the invention. All headings used herein are for convenience only. All patents and publications mentioned in the specification are indicative of the levels of skill of those skilled in the art to which the invention pertains, and are herein incorporated by reference to the same extent as if each individual publication, patent or patent application was specifically and individually indicated to be incorporated by reference. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicants' invention, including compounds for which an enabling disclosure is provided in the references cited herein, are not intended to be included in the composition of matter claims herein.

We claim:

1. A metallized nucleic acid ring containing one or more metallized segments and one or more noncontiguous unmetallized segments wherein the nucleic acid ring is at least 50 nm in circumference.

2. The metallized nucleic acid ring of claim 1 wherein the ring is metallized with silver or gold.

3. The metallized nucleic acid ring of claim 1 wherein the nucleic acid is single stranded or double stranded nucleic acid.

4. The metallized nucleic acid ring of claim 1 wherein the nucleic acid is DNA.

5. The metallized nucleic acid ring of claim 1 wherein the nucleic acid is RNA.

6. The metallized nucleic acid ring of claim 1 further comprising an insulating layer which coats the metallized nucleic acid rings, said layer comprising a self-assembled monolayer.

7. The metallized nucleic acid ring of claim 6 wherein the self-assembled monolayer comprises an alkanethiol.

8. A negative refractive index material comprising a plurality of metallized nucleic acid rings, wherein each ring contains one or more metallized segments and one or more noncontiguous unmetallized segments, wherein said material has a negative electric permittivity and a negative magnetic permeability.

9. The negative refractive index material of claim 8 wherein the circumferences of the metallized nucleic acid rings are the same.

10. The negative refractive index material of claim 8 wherein the circumferences of at least a portion of the metallized nucleic acid rings are different.

11. The negative refractive index material of claim 8 comprising stacks of two or more of said metalized nucleic acid rings.

12. The negative refractive index material of claim 11 wherein the nucleic acid rings comprise single strand DNA molecules.

13. The negative refractive index material of claim 11 wherein the nucleic acid rings comprise double strand DNA molecules.

14. The negative refractive index material of claim 11 wherein the nucleic acid rings comprise RNA molecules.

15. The negative refractive index material of claim 11 wherein each nucleic acid ring has a circumference between approximately 50 nm and approximately 1000 nm.

16. The negative refractive index material of claim 11 wherein each nucleic acid ring has a circumference between approximately 60 nm and approximately 320 nm.

17. The negative refractive index material of claim 11 further comprising an insulating layer which coats the metallized nucleic acid rings, said layer comprising a self-assembled monolayer.

18. The negative refractive index material of claim 17 wherein the self-assembled monolayer is an alkanethiol.

19. The negative refractive index material of claim 11 wherein the nucleic acid rings are metallized with silver or a silver containing composition.

20. A method of making a negative refractive index material comprising:
a) linearly attaching one or more metallizable nucleic acid segments to one or more non-metallizable segments to form a linear nucleic acid element;
b) joining the ends of the linear nucleic acid element to each other to form a circular split-ring template;
c) connecting two or more of said split-ring templates together in parallel; and
d) metallizing two or more split-ring templates so that the metallizable nucleic acid segments are coated with a conductive metal while the one or more non-metallizable segments remain uncoated by the metal.

21. The method of claim 20 where said conductive metal is silver or a silver containing composition.

22. The method of claim 20 wherein said one or more non-metallizable segments are hydrophobic organic spacers.

23. The method of claim 20 further comprising attaching dendrimers to the one or more non-metallizable segments prior to metallizing two or more split-ring templates.

24. The method of claim 20 wherein said one or more non-metallizable segments are inert nucleic acid sequences.

25. The method of claim 20 wherein said one or more metallizable nucleic acid segments are single strand or double strand DNA molecules.

26. The method of claim 20 wherein said one or more metallizable nucleic acid segments are RNA molecules.

27. The method of claim 20 wherein each linear nucleic acid element has a length between approximately 50 nm and approximately 1000 nm.

28. The method of claim 20 wherein each linear nucleic acid element has a length between approximately 60 nm and approximately 320 nm.

29. The method of claim 20 further comprising coating the metallized two or more split-ring templates with an insulating layer.

30. The method of claim 20 wherein said two or more split-ring templates are connected together to have an inter-ring axial spacing closer than the radial metal thickness.
